(19) United States
(12) Patent Application Publication
(10) Pub. No.: US 2023/0346252 A1
(43) Pub. Date: Nov. 2, 2023

(54) SYSTEMS AND METHODS FOR DETERMINING A RESPIRATORY EFFORT OF A PATIENT

(71) Applicant: TIMPLE MEDICAL B.V., Eindhoven (NL)

(72) Inventor: Maria Aparecida Miyuki Nakamura, Sao Paulo (BR)

(21) Appl. No.: 17/766,918

(22) PCT Filed: Oct. 7, 2020
(86) PCT No.: PCT/IB2020/059439

§ 371 Date: Apr. 6, 2022

Related U.S. Application Data
(60) Provisional application No. 62/916,929, filed on Oct. 18, 2019.

(51) Int. Cl. A61B 5/08; A61B 5/0536; A61B 5/087

(52) U.S. Cl. CPC A61B 5/0809; A61B 5/0536; A61B 5/0806; A61B 5/087; A61M 2205/505; A61M 2205/52

(57) ABSTRACT

A method of determining a patient's respiratory effort and related patient-ventilator asynchrony comprises acquiring first impedance data representative of a first region of the lungs of the patient, the first region comprising at least a dependent region of the lungs, during the applied positive expiratory pressure, optionally acquiring second impedance data representative of a second region of the lungs, and comparing the first impedance data with one or more of the second impedance data, a flow rate within a breathing circuit of the patient, a pressure within the breathing circuit, historical impedance data of the first region, and stored patterns of impedance data of the first region. Related systems for determining a respiratory effort of a patient are also disclosed.

### PRIORITY CLAIM

This application is a national phase entry under 35 U.S.C. § 371 of International Patent Application PCT/M2020/059439, filed Oct. 7, 2020, designating the United States of America and published as International Patent Publication WO 2021/074747 A1 on Apr. 22, 2021, which claims the benefit under Article 8 of the Patent Cooperation Treaty to U.S. Provisional Patent Application Ser. No. 62/916,929, filed Oct. 18, 2019, for “SYSTEMS AND METHODS FOR DETERMINING A RESPIRATORY EFFORT OF A PATIENT.”

### TECHNICAL FIELD

Embodiments of the disclosure relate generally to systems and methods of determining a respiratory effort of a patient. More particularly, embodiments of the disclosure relate to methods of determining respiratory efforts of a patient and/or patient-ventilator asynchronies, such as an ineffective effort, based at least on impedance data and/or information derived from impedance data of a dependent region of the lungs, and to related devices and systems.

### BACKGROUND

The treatment of patients with respiratory failure requires artificial ventilator support. Patients with respiratory failure, such as patients with acute respiratory distress syndrome (ARDS), may be exposed to artificial ventilator support and positive end expiratory pressure (PEEP). Unfortunately, sometimes the patient and the mechanical ventilator can become out of synchrony. Patient-ventilator asynchrony is defined by a poor interaction between the patient and the ventilator. Patient-ventilator asynchrony affects 35-43% of mechanically ventilated patients and it has been associated with poor outcomes for the patient, such as prolonged mechanical ventilation and higher in-hospital mortality rates. One result of patient-ventilator asynchrony is ineffective inspiratory efforts (also referred to as “ineffective efforts”) that occur when a patient effort to initiate a breath is unrecognized by the mechanical ventilator and the mechanical ventilator is unresponsive to the patient's attempt to breathe. Ineffective inspiratory efforts during expiration (IEE) are defined as inspiratory efforts (by the patient) that are unable to trigger a ventilator-delivered breath, and are the most common type of patient-ventilator asynchrony. The exact incidents of IEE vary and depend on several factors, such as the particular patient, mechanical ventilation (MV) settings, sedation level, and wakefulness state of the patient.

Unfortunately, despite the importance of detecting the patient's effort, and harmful effect of asynchrony on patient outcome, it is difficult to detect the patient's effort and asynchrony based only on the ventilator waveforms, even by experts. Typically, patient's effort is detected with invasive techniques, such as with an esophageal catheter or measurements of electrical activity of the diaphragm (Eadi measurements), in order to reduce the incidence of Ineffective Effort Asynchrony. For example, the patient's effort may be detected with esophageal pressure tracings, with an esophageal balloon, or with neutrally adjusted ventilator assist (NAVA). However, both esophageal pressure and NAVA monitoring require the positioning of a catheter, an invasive and complex procedure.

### BRIEF SUMMARY

In some embodiments, a method of determining a respiratory effort of a patient comprises acquiring first impedance data representative of a first region of lungs of a patient, the first region comprising at least a dependent region of the lungs, comparing the first impedance data with one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region, and based on the comparison, identifying the occurrence of patient's respiratory effort.

In additional embodiments, a system for determining a respiratory effort of a patient comprises an electrical impedance tomography system, at least one processor coupled to the electrical impedance tomography system, and at least one non-transitory computer readable storage medium storing instructions thereon that, when executed by the at least one processor, cause the at least one processor to acquire, with the electrical impedance tomography system, first impedance data representative of at least one dependent region of lungs of a patient, and compare the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs to identify a respiratory effort of the patient based on the comparison.

In yet other embodiments, a system for determining a respiratory effort of a patient comprises an electrical impedance tomography system, and a controller, wherein the electrical impedance tomography system is operably coupled to the controller. The controller comprises at least one processor, and at least one non-transitory computer readable storage medium storing instructions thereon that, when executed by the at least one processor, cause the at least one processor to cause the electrical impedance tomography system to acquire first impedance data of a first region of lungs of the patient, the first region comprising a dependent region of the lungs, compare the first impedance data with one or more of second impedance data representative of a non-dependent region of the lungs of the patient, a flow rate within a breathing circuit of the patient, a pressure within the breathing circuit, a historical impedance data of the first region, and stored patterns of impedance data of the first region, and identify a respiratory effort of the patient based on the comparison.

### BRIEF DESCRIPTION OF THE DRAWINGS

FIG. 1 is a schematic diagram of a portion of an Electrical Impedance Tomography (EIT) system showing a plurality of electrodes positioned around a region of interest of a patient, according to one or more embodiments of the present disclosure;

FIG. 2 is a schematic diagram showing a cross-section of the thorax of the patient along the plane of the electrodes according to one or more embodiments of the present disclosure;

FIG. 3 is a schematic block diagram of an EIT system, according to an embodiment of the disclosure;

FIG. 4 is a schematic view of system for identifying a patient's respiratory effort and a patient-ventilator asynchrony, according to one or more embodiments of the disclosure;

FIG. 5 is a flow chart of a method of determining a respiratory related effort of a patient, according to one or more embodiments of the disclosure;

FIG. 6A is a graph of flow and airway pressure measured at the patient's airway, and impendence data of the patient's lungs (Delta Z), measured at the posterior and at the anterior regions, according to embodiments of the disclosure;

FIG. 6B is an EIT image illustrating a dependent region and a non-dependent region of the lungs of a patient, according to embodiments of the disclosure;

FIG. 6C is a graph of flow and airway pressure measured at the patient's airway, and impendence data of the patient's lungs, measured at the posterior and at the anterior regions, according to embodiments of the disclosure;

FIG. 6D is a graph of flow and airway pressure measured at the patient's airway, and impendence data of the patient's lungs, measured at the posterior and at the anterior regions, according to embodiments of the disclosure; and

FIG. 7 is a graph of flow and airway pressure measured at the airway of a piglet, and impendence data of the lungs, measured at the posterior and at the anterior regions, as well as graphs of the electrical activity of the diaphragm.

### DETAILED DESCRIPTION

This provisional patent application is related to Appendix A and Appendix B, each of which are attached hereto, which also form a part of the present disclosure. The published documents referenced in the References section at the end of Appendix A are hereby incorporated herein by this reference.

Illustrations presented herein are not meant to be actual views of any particular material, component, or system, but are merely idealized representations that are employed to describe embodiments of the disclosure.

The following description provides specific details, such as material types, dimensions, and processing conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional fabrication techniques employed in the industry. In addition, the description provided below does not form a complete process flow, apparatus, or system for determining a respiratory effort of a patient, a patient-ventilator asynchrony, or a related method. Only those process acts and structures necessary to understand the embodiments of the disclosure are described in detail below. Additional acts to determine a respiratory effort of a patient or a patient-ventilator asynchrony may be performed by conventional techniques. Further, any drawings accompanying the present application are for illustrative purposes only and, thus, are not drawn to scale. Additionally, elements common between figures may retain the same numerical designation.

In the following detailed description, reference is made to the accompanying drawings, which form a part hereof, and in which is shown by way of illustration specific embodiments in which the disclosure may be practiced. These embodiments are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

In accordance with common practice, the various features illustrated in the drawings may not be drawn to scale. The illustrations presented herein are not meant to be actual views of any particular apparatus (e.g., device, system, etc.) or method, but are merely representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus or all operations of a particular method.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof. Some drawings may illustrate signals as a single signal for clarity of presentation and description. It should be understood by a person of ordinary skill in the art that the signal may represent a bus of signals, wherein the bus may have a variety of bit widths and the disclosure may be implemented on any number of data signals including a single data signal.

The various illustrative logical blocks, modules, and circuits described in connection with the embodiments disclosed herein may be implemented or performed with a general purpose processor, a special purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A general-purpose processor may be considered a special-purpose processor while the general-purpose processor executes instructions (e.g., software code) stored on a computer-readable medium. A processor may also be implemented as a combination of computing devices, e.g., a combination of a DSP and a microprocessor, a plurality of microprocessors, one or more microprocessors in conjunction with a DSP core, or any other such configuration.

Also, it is noted that embodiments may be described in terms of a process that may be depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, etc. Furthermore, the methods disclosed herein may be implemented in hardware, software, or both. If implemented in software, the functions may be stored or transmitted as one or more instructions or code on computer-readable media. Computer-readable media include both computer storage media and communication media, including any medium that facilitates transfer of a computer program from one place to another.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, a “dependent” region of the lungs means and includes a lower region of the lungs, such as a region of the lungs located proximate a back of a patient (as opposed to regions of the lungs that are located proximate a chest of the patient). The dependent region of the lungs may include the dorsal region, which may also be referred to as posterior region of the lungs.

As used herein, a “non-dependent” region of the lungs means and includes an upper region of the lungs, such as a region of the lungs located proximate a chest of a patient. The non-dependent regions of the lungs may include the ventral region, which may also be referred to as the anterior region of the lungs.

As used herein, the term “impedance data” refers to a signal from an Electrical Impedance Tomography system and also includes information that may be derived from the signal of the Electrical Impedance Tomography system. Non-limiting examples of the information that may be derived from the signal of the Electrical Impedance Tomography system include images (EIT images), a plethysmograph derived from the EIT data, chord compliance, and perfusion.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, the term “substantially” in reference to a given parameter, property, or condition means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or even at least about 99% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

According to embodiments described herein, a patient's respiratory effort may be determined using non-invasive techniques. In some embodiments, a patient-ventilator asynchrony such as ineffective effort (IEE; also referred to as “ineffective inspiratory effort”) may be detected by comparing impedance data (e.g., an EIT plethysmograph) of at least one dependent region of the lungs to one or more of historical impedance data of the at least one dependent region of the lungs, impedance data of a non-dependent region of the lungs, airway pressure, airway flow, and stored patterns of impedance data of the at least one dependent region. In some embodiments, responsive to determining a relative increase in the impedance of the dependent region of the lungs at a time without a corresponding increase in the impedance of the non-dependent region of the lungs, an increase in the flow rate in the airway, a pressure in the airway, the historical impedance data of the at least one dependent region, or the stored pattern of impedance of the at least one dependent region, a respiratory effort of the patient (such as a patient-ventilator asynchrony) may be detected. Each of the impedance of the dependent region, the impedance data of the non-dependent region, the flow rate in the airway, the pressure in the airway, the historical impedance data of the at least one dependent region, and the stored pattern of impedance data of the at least one dependent region may be determined non-invasively. Responsive to detecting the patient-ventilator asynchrony, a corrective action may be taken, such as adjusting ventilator parameters in order to reduce the incidence of the asynchrony. Related systems and devices for detecting the patient-ventilator asynchrony are also disclosed. Accordingly, a respiratory effort of a patient and/or a patient-ventilator asynchrony may be detected with non-invasive methods, such as with an EIT system a mechanical ventilator.

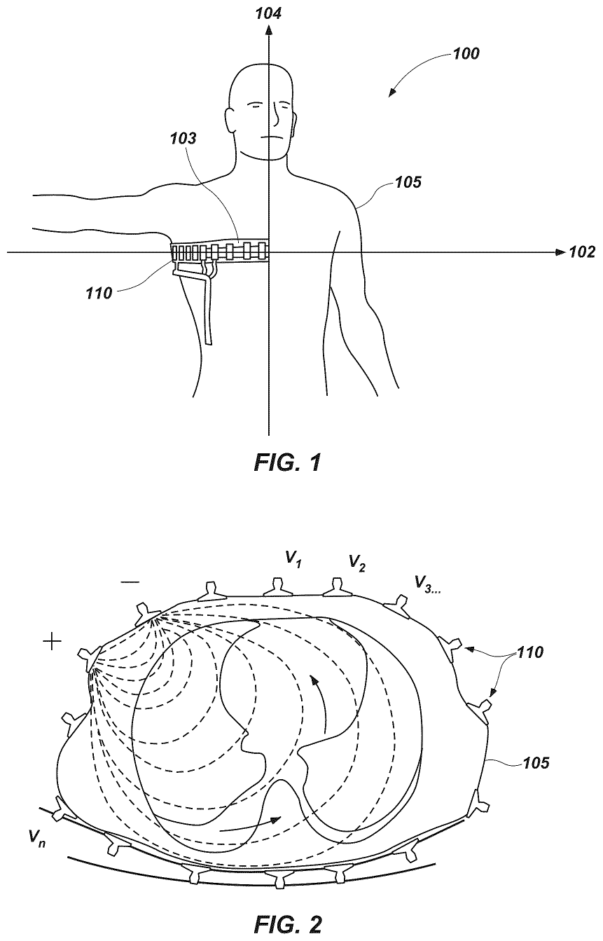

FIG. 1 is a schematic diagram of a portion of an EIT system 100 showing a plurality of electrodes 110 positioned around a region of interest (e.g., thorax) of a patient 105. The electrodes 110 of conventional EIT systems 100 are typically physically held in place by an electrode belt 103. The placement of the electrodes 110 is typically transverse to the cranial caudal axis 104 of the patient and substantially parallel to axis 102. Although the electrodes 110 are shown in FIG. 1 as being placed only partially around the patient 105, electrodes 110 may by placed around the entire patient 105 depending on the specific region of interest available or desired for measurement. The electrodes 110 may be coupled to a computing system (not shown) configured to control the operation of the electrodes 110 and perform reconstruction of the EIT image.

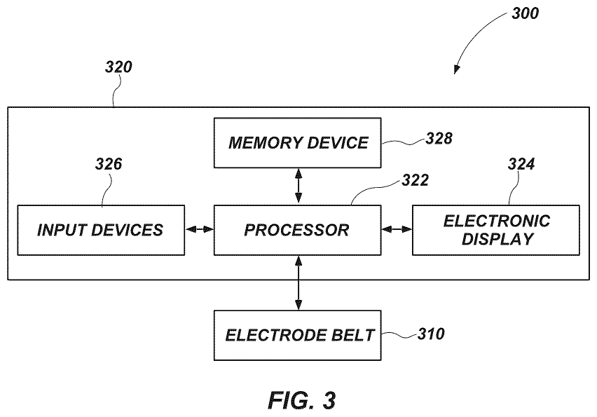

FIG. 2 is a schematic diagram showing a cross-section of the thorax of the patient 105 along the plane of the electrodes. A voltage may be applied to a pair of electrodes 110 (shown by the electrodes having a + and − symbol) to inject an excitation current into the patient between an electrode pair. As a result, voltages (e.g., V₁, V₂, V₃ . . . Vₙ) may be detected by the other electrodes and measured by the EIT system 100. Current injection may be performed for a measurement cycle according to a circular pattern using different electrode pairs to generate the excitation current.

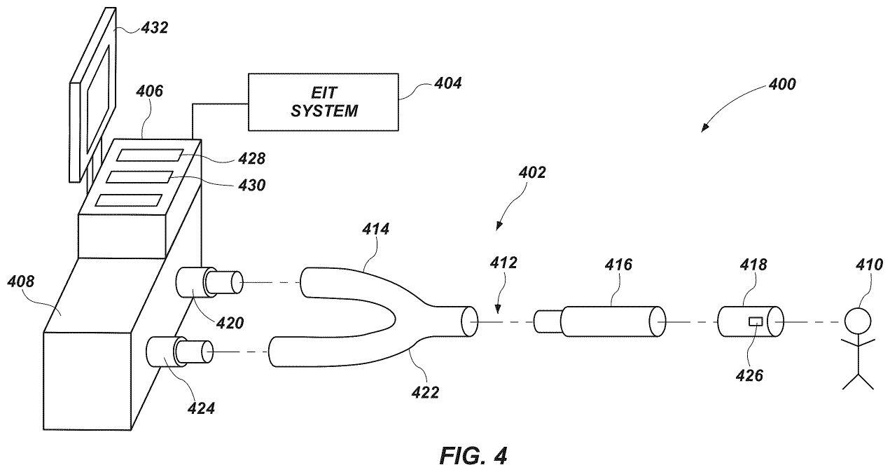

FIG. 3 is a schematic block diagram of an EIT system 300 according to an embodiment of the disclosure. The EIT system 300 may include an electrode belt 310 operably coupled with a data processing system 320. The electrode belt 310 and the data processing system 320 may be coupled together via a wired connection (e.g., cables) and/or may have communication modules to communicate wirelessly with each other. The data processing system 320 may include a processor 322 operably coupled with an electronic display 324, input devices 326, and a memory device 328. The electronic display 324 may be constructed with the data processing system 320 into a singular form factor for an EIT device coupled with the electrode belt 310. In some embodiments, the electronic display 324 and the data processing system 320 may be separate units of the EIT device coupled with the electrode belt 310. In yet other embodiments, an EIT system 300 may be integrated within another host system configured to perform additional medical measurements and/or procedures, in which the electrode belt 310 may couple to a port of the host system already having its own input devices, memory devices, and electronic display. As such, the host system may have the EIT processing software installed therein. Such software may be built into the host system prior field use or updated after installation.

The processor 322 may coordinate the communication between the various devices as well as execute instructions stored in computer-readable media of the memory device 328 to direct current excitation, data acquisition, data analysis, and/or image reconstruction. As an example, the memory device 328 may include a library of finite element meshes used by the processor 322 to model the patient's body in the region of interest for performing image reconstruction. In some embodiments, the memory device 328 includes historical data of the impedance of a patient's lungs (such as the impedance data of a region of interest (e.g., a dependent region) and impedance data of other regions of the lungs) and/or patterns of impedance data of the patient's lungs. Input devices 326 may include devices such as a keyboard, touch screen interface, computer mouse, remote control, mobile devices, or other devices that are configured to receive information that may be used by the processor 322 to receive inputs from an operator of the EIT system 300. Thus, for a touch screen interface the electronic display 324 and the input devices 326 receiving user input may be integrated within the same device. The electronic display 324 may be configured to receive the data and output the EIT image reconstructed by the processor for the operator to view. Additional data (e.g., numeric data, graphs, trend information, and other information deemed useful for the operator) may also be generated by the processor 322 from the measured EIT data alone, or in combination with other non-EIT data according to other equipment coupled thereto. Such additional data may be displayed on the electronic display 324.

The EIT system 300 may include components that are not shown in the figures, but may also be included to facilitate communication and/or current excitation with the electrode belt 310 as would be understood by one of ordinary skill in the art, such as including one or more analog to digital converter, signal treatment circuits, demodulation circuits, power sources, etc.

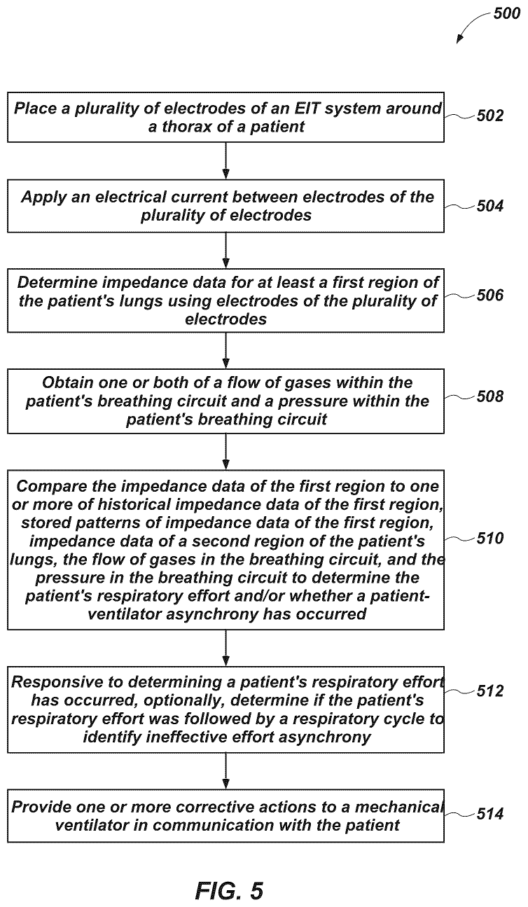

FIG. 4 is a schematic view of system for identifying a patient's respiratory effort and a patient-ventilator asynchrony. For instance, FIG. 4 depicts a system 400 for determining if a given patient 410 connected to a mechanical ventilator 408 is exhibiting an asynchrony with the mechanical ventilator 408. The system 400 may include a ventilator system 402, an EIT system 404, and a controller 406 for operating the system 400 and specifically, the ventilator system 402, an EIT system 404. The ventilator system 402 and the EIT system 404 are operably coupled to the controller 406.

In some embodiments, the ventilator system 402 includes the mechanical ventilator 408 that provides respiratory support or respiratory assistance to the patient 410. For instance, the mechanical ventilator 408 may provide a flow of medical gas, which may include one or more of air, oxygen, nitrogen, and helium. In some embodiments, the flow of medical gas may further include additives such as aerosol drugs or anesthetic agents. The ventilator system 402 may further include a breathing circuit 412, an inspiratory limb 414, a patient limb 416, and a patient connection 418. In some embodiments, the mechanical ventilator 408 may provide a flow of medical gas to the breathing circuit 412 through the inspiratory limb 414, which is connected to an inspiratory port 420 of the mechanical ventilator 408. The medical gas may flow (e.g., travel) through the inspiratory limb 414 and into the patient limb 416 of the breathing circuit 412. Accordingly, the mechanical ventilator 408 may provide the medical gas to the patient 410 through the patient connection 418.

Expired gases from the patient 410 may be delivered back to the mechanical ventilator 408 through the patient connection 418 and the patient limb 416. In some embodiments, the expired gases may be directed into an expiratory limb 422 of the breathing circuit 412 via one or more valves (e.g., check valves). For instance, the ventilator system 402 may further include a plurality of check valves, which may be placed at various points along the breathing circuit 412 such as to only permit medical gas flow in a desired direction along the appropriate pathway toward or away from the patient 410.

Additionally, the expired gases may be returned to the mechanical ventilator 408 through an expiratory port 424 of the mechanical ventilator 408.

In some embodiments, the expiratory port 424 may include a controllable flow valve that is adjustable to regulate the pressure within the breathing circuit 412. Adjusting the flow valve may create a back pressure, which is applied to the patient 410 during exhalation to create a positive end expiratory pressure. Accordingly, the system 400 may include any conventional system for providing PEEP therapy to a patient 410. Additionally, other systems and configurations as recognized by one of ordinary skill in the art fall within the scope of the present disclosure.

The ventilator system 402 may further include one or more gas monitoring sensors 426. In some embodiments, the one or more gas monitoring sensors 426 may be disposed within the patient connection 418 of the breathing circuit 412. In alternative embodiments, the one or more gas monitoring sensors 426 may be fluidly connected to any other component of the breathing circuit 412 or the ventilator system 402. In some embodiments, the gas monitoring sensor 426 may include one or more of a pressure, a flow, and a gas concentration sensor. As is described in further detail below, the controller 406 and the mechanical ventilator 408 may utilize the one or more gas monitoring sensors 426 to monitor and ultimately, control the operation of the mechanical ventilator 408 and provide information (e.g., feedback to a user (e.g., clinician)). In some embodiments, the one or more gas monitoring sensors 426 may include any conventional gas sensors.

The EIT system 404 may include any of the EIT systems described above in regard to FIG. 1 through FIG. 3 and may operate according to any of the embodiments described above. In some embodiments, the EIT system 404 may include any conventional EIT system. Additionally, as noted above, the EIT system 404 may be operably coupled to the controller 406 and may provide information to controller 406 regarding measurements performed by the EIT system 404. In some embodiments, the EIT system 404 may be completely independent of the ventilator system 402 and may determine that the PEEP was changed during a maneuver through via a respective pressure sensor operably coupled to the EIT system 404. In one or more embodiments, the EIT system 404 may also have a respective electronic display and input devices separate from displays and/or input devices of the ventilator system 402. Furthermore, the electronic display and input devices of the EIT system 404 may be utilized to input (e.g., manually input) information about the PEEP and/or other ventilatory parameters.

The controller 406 may include a processor 428 coupled to a memory 430 and an input/output component 432. The processor 428 may comprise a microprocessor, a field-programmable gate array, and/or other suitable logic devices. The processor 428 may execute instructions stored in computer-readable media of the memory device 430 to direct current excitation, data acquisition, data analysis, image reconstruction, and/or determination of a patient-ventilator asynchrony. The memory 430 may include volatile and/or nonvolatile media (e.g., ROM, RAM, magnetic disk storage media, optical storage media, flash memory devices, and/or other suitable storage media) and/or other types of computer-readable storage media (e.g., a non-transitory computer-readable storage medium) configured to store data. The memory 430 may store algorithms and/or instructions for operating the ventilator system 402 and the EIT system 404, to be executed by the processor 428. For example, the controller 406 may include the data processing system 320 described above in regard to FIG. 3. In some embodiments, the processor 428 is operably coupled to send data to a computing device operatively coupled (e.g., over the Internet) to the controller 406, such as a server or personal computer. The input/output component 432 can include a display, a touch screen, a keyboard, a mouse, and/or other suitable types of input/output devices configured to accept input from and provide output to an operator. In some embodiments, the memory 430 may include historical data of the impedance of a patient's lungs (such as the impedance data of a region of interest (e.g., a dependent region)) and impedance data of other regions of the lungs.

Referring still to FIG. 4, the system 400 may utilize the ventilator system 402 to apply ventilatory support and may be configured to determine the presence of a patient's respiratory effort and patient-ventilator asynchrony, such as ineffective effort. In some embodiments, the system 400 is configured to provide levels of PEEP to the patient based on a determined respiratory effort and/or patient-ventilator asynchrony. PEEP increases a base line pressure within the patient's respiratory system such that natural exhalation by the patient maintains a higher airway pressure than respiration without PEEP therapy. Conventional PEEP pressures range from 0 up to 40 cm H₂O, although higher PEEP pressures may also be used.

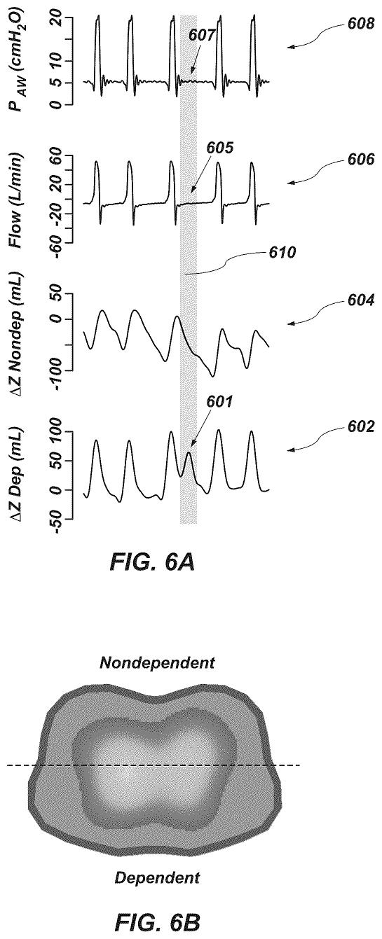

In some embodiments, an ineffective effort may be determined using the EIT system 300, 404, and/or the system 400. FIG. 5 is a simplified flow diagram illustrating a method 500 of determining a respiratory related effort of a patient. The method 500 may include placing a plurality of electrodes of an EIT system (e.g., the EIT system 100) around a thorax of a patient; act 504 including applying an electrical current between electrodes of the plurality of electrodes; act 506 including determining impedance data for at least one first region of the patient's lungs using electrodes of the plurality of electrodes; act 508 including obtaining one or both of a flow of gases within the patient's breathing circuit and a pressure within the patient's breathing circuit; act 510 including comparing the impedance data of the first region to one or more of historical impedance data of the first region, stored patterns of the impedance data of the first region, impedance data of the at least a second region of the patient's lungs, the flow of gases in the breathing circuit, and the pressure within the patient's breathing circuit to determine a patient's respiratory effort and/or whether a patient-ventilator asynchrony has occurred, act 512 including, responsive to determining that a patient respiratory effort has occurred, optionally determine if the patient's respiratory effort was followed by a respiratory cycle to identify an ineffective effort asynchrony; and act 514 including providing one or more corrective actions to a mechanical ventilator in communication with the patient.

Act 502 includes placing a plurality of electrodes of an EIT system around the thorax of a patient. In some embodiments, act 502 includes operably connecting the patient to a mechanical ventilator. In some embodiments, the EIT system comprises any of the EIT systems described above (the EIT system 100, the EIT system 300, the EIT system 404) and the patient is connected to (in communication with) a mechanical ventilator, such as the mechanical ventilator 408 of FIG. 4. However, the disclosure is not so limited and the patient may be connected to a mechanical ventilator.

Act 504 includes applying an electrical current between electrodes of the plurality of electrodes. In some embodiments, voltages are applied sequentially between a pair of electrodes to inject an excitation current into the patient between the electrode pair and voltages may be measured with other electrodes of the EIT system. Current injection may be performed for a measurement cycle according to a circular pattern using different electrode pairs to generate the excitation current, as described above with reference to FIG. 2. In some embodiments, act 504 occurs simultaneously with the provision of mechanical ventilation to a patient, such as via PEEP therapy. However, the disclosure is not so limited and act 504 may occur without the provision of mechanical ventilation to the patient.

Act 506 includes determining impedance data for a first region of the patient's lungs using electrodes of the plurality of electrodes. In some embodiments, act 506 further includes determining impedance data for at least a second region of the patient's lungs. In some embodiments, determining impedance data for the at least a second region of the patient is performed substantially concurrently with determining impedance data for the first region of the patient's lungs using electrodes of the plurality of electrodes. In some such embodiments, the voltage data (corresponding to the impedance data) for the first region and the at least a second region are measured concurrently.

In some embodiments, the first region of the patient's lungs comprises a dependent (e.g., dorsal, posterior) region of the patient's lungs. The dependent region may include portions of the lungs proximate the patient's back. In other words, the dependent region comprises portions of the lungs located closer to the floor when the patient is lying prone and facing upwards. In some embodiments, the first region includes at least one dependent region. In some embodiments, the first region includes only dependent regions of the lungs of the patient. However, the disclosure is not so limited and in other embodiments, the first region includes dependent regions and non-dependent regions of the lungs of the patient. The first region may be referred to herein as an “Ineffective Effort Region of Interest” (IEROI).

The at least one second region may include at least one region of the lungs of the patient different from the first region. In some embodiments, the at least one second region comprises a non-dependent (e.g., ventral, anterior) region of the lungs of the patient. The non-dependent region may include portions of the lungs of the patient proximate a chest (and distal from a back) of the patient. In other words, the non-dependent region comprises portions of the lungs located distal from the floor when the patient is lying prone and facing upwards. In some embodiments, the at least a second region is free of (does not include) dependent regions of the lungs and does not include an IEROI.

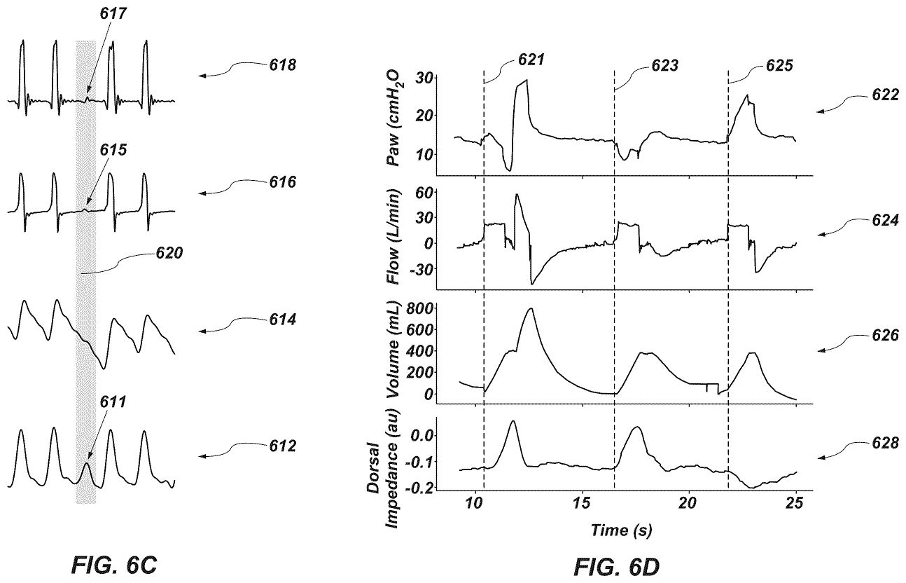

FIG. 6A is a graph of flow and airway pressure measured at a patient's airway, and impedance data of the patient's lungs (Delta Z) measured at the posterior and anterior regions, according to embodiments of the disclosure. In FIG. 6A, graph 602 represents the EIT plethysmograph of the first region of the lungs, which may comprise a dependent region of the lungs and includes an IEROI. Graph 604 represents the EIT plethysmograph of the second region of the lungs over the same period of time as the EIT plethysmograph of the first region of the lungs. Graph 606 represents the flow of gases within the patient's breathing circuit and graph 608 represents the pressure within the patient's breathing circuit over the same period of time. The flow of gases and the pressure within the patient's breathing circuit may be measured with a gas monitoring sensor (e.g., the gas monitoring sensor 426 (FIG. 4)), with a ventilator (e.g., the mechanical ventilator 408 (FIG. 4)), or both. Each of the EIT plethysmograph of the first region (the first impedance data), the EIT plethysmograph of the at least a second region (the second impedance data), the flow rate, and the pressure within the patient's breathing circuit may be measured substantially concurrently, as illustrated in FIG. 6A, wherein the x-axis represents the time of the respective measurements.

The EIT plethysmograph of the first region and the EIT plethysmograph of the at least a second region may be obtained simultaneously and during ventilator support and PEEP therapy, such as during ascending and descending PEEP steps for recruitment of alveoli and/or lung recruitment, as described in U.S. Patent Application Publication No. 2019/0246949, the entire disclosure of which is hereby incorporated herein by this reference. The EIT plethysmograph of the first region and the EIT plethysmograph of the at least a second region may represent a sum of the underlying impedance of pixels of an EIT image of the respective first region and the at least a second region of the lungs within an EIT image. For example, with reference to FIG. 6B, an EIT image illustrating a dependent region and a non-dependent region of the lungs of a patient is illustrated. A lower half of FIG. 6B may represent a dependent region of the lungs and an upper half may represent non-dependent region of the lungs. The plethysmographs may represent a sum of impedances in a certain region of interest, and may also be derived from values of the pixels determined after an image reconstruction algorithm is applied to the impedance data.

With reference again to FIG. 5 and FIG. 6A, act 506 includes measuring the impedance data within the first region. In some embodiments, act 506 further comprises measuring the impedance data within a second region of the lungs over the course of time. In some embodiments, the measured impedance data is represented as a relative change in impedance of the respective first region and the second region of the lungs (such as with reference to a reference baseline impedance in each of the first region and the second region). In some embodiments, the reference baseline represents an average of voltages sets acquired during an initial duration of with EIT system.

Referring back to FIG. 5, act 508 includes obtaining one or both of a flow of gases within the patient's breathing circuit and a pressure within the patient's breathing circuit. In some embodiments, obtaining the flow of gases and the pressure within the patient's breathing circuit comprises measuring the flow of gases and the pressure. In other embodiments, the flow of gases and the pressure is obtained by a ventilator, which may control the flow of gases and the pressure. In some embodiments, the flow of gases within the patient's breathing circuit may be measured with a gas monitoring sensor, such as the gas monitoring sensor 426 (FIG. 4). The gas monitoring sensor may be placed anywhere within the patient's breathing circuit. The pressure within the patient's breathing circuit may be measured with a gas monitoring sensor, which may comprise the same gas monitoring sensor or a different gas monitoring sensor than the gas monitoring sensor that measures the flow of gases. In some embodiments, one or both of the flow of gases or the pressure within the patient's breathing circuit may be measured with a ventilator, such as the mechanical ventilator 408 (FIG. 4).

Act 510 may include comparing the impedance data of the first region to one or more of historical impedance data of the first region, stored patterns of the impedance data of the first region, impedance data of the second region, the flow of gases in in the patient's breathing circuit, and the pressure in the patient's breathing circuit to determine a patient's respiratory effort and/or whether a patient-ventilator asynchrony has occurred. For example, with reference back to FIG. 6A, the first impedance data of the first region of the lungs (graph 602) may be compared to one or more of the impedance data of a second region of the lungs (graph 604), the flow rate within the breathing circuit (graph 606), and the pressure within the breathing circuit (graph 608). Shaded area 610 of FIG. 6A represents a time frame within which an ineffective effort asynchrony has occurred. During the time period within the shaded region 610, the impedance within the first region (dependent region) exhibits an increase, indicated at arrow 601 without a corresponding increase in the impedance of a second region (non-dependent region), as indicated in graph 604, without an increase in flow, indicated at arrow 605, and without an increase in airway pressure, as indicated at arrow 607. In other words, the impedance within the second region does not exhibit a corresponding increase as the impedance within the first region. In addition, during the same time period, the flow and the pressure within the airway also do not exhibit a change, as occurs during normal breathing cycles. Accordingly, the patient's respiratory effort and a patient-ventilator asynchrony (e.g., ineffective effort) may be determined by comparing the impedance data of the first region to one or more of the impedance data of the at least a second region, the flow rate within the airway, and the pressure within the airway. In additional embodiments, the patient-ventilator asynchrony may be determined based on a comparison of the impedance of the first region relative to historical values of the impedance of the first region, to stored patterns of impedance data of the first region, or both. In some embodiments, the patient-ventilator asynchrony may be determined based on a comparison of the impedance of the first region to the impedance of the first region during a previous normal breathing cycle or an ineffective effort cycle.

Act 512 may include, responsive to determining that a patient respiratory effort has occurred, optionally determining if the patient's respiratory effort was followed by a respiratory cycle to identify an ineffective effort asynchrony.

Act 514 includes providing one or more corrective actions to a mechanical ventilator in communication with the patient responsive to identifying an ineffective effort asynchrony. In some embodiments, the one or more corrective actions includes changing ventilator parameters such as PEEP, I:E (inspiratory:expiratory, also referred to as a ventilation ratio) relationship, Respiratory Rate, and Trigger settings, and providing an indication of the patient-ventilator asynchrony (such as on the input/output component 432 (FIG. 4)).

FIG. 6C is a graph of flow and airway pressure measured at the patient's airway, and impendence data of the patient's lungs, measured at the posterior and at the anterior regions, according to embodiments of the disclosure. The graph illustrates each of a flow rate of gases to the patient within the patient's breathing circuit, a pressure within the patient's breathing circuit, an EIT plethysmograph measured over time in a first region of the lungs, and an EIT plethysmograph measured over time in the at least a second region of the lung over the same time range during an ineffective effort asynchrony. Graph 612 represents the EIT plethysmograph of the first region of the lungs, which may comprise a dependent region of the lungs and includes an IEROI, graph 614 represents the EIT plethysmograph of the second region of the lungs over the same period of time as the EIT plethysmograph of the first region of the lungs, graph 616 represents the flow of gases within the patient's breathing circuit and graph 618 represents the pressure within the patient's breathing circuit.

The ineffective respiratory effort of the patient occurs during the time period corresponding to the shaded region 620. Arrow 611 indicates a minor increase in the impedance within the first region without a corresponding increase in the impedance within the at least a second region. The second region shows a reduction in impedance just after the shaded region 620, indicating a drop in air content in the at least second region. Arrow 615 and arrow 617, respectively, indicate that the flow or pressure do not increase at a time corresponding to the minor increase in impedance within the first region (arrow 611), confirming that the change (increase) in impedance within the first region is in response to a patient's effort, which did not cause the trigger of a ventilatory cycle, indicating a patient-ventilator asynchrony (ineffective effort). In other words, the change (increase) in impedance within the first region is in response to the respiratory effort of the patient and such respiratory effort did not trigger a ventilator cycle, as indicated in the flow and pressure curves, resulting in a patient-ventilator asynchrony.

FIG. 6D is a graph of flow and airway pressure measured at the airway of the patient, and impendence data of the patient's lungs, measured at the posterior and at the anterior regions, according to embodiments of the disclosure. In FIG. 6D, graph 622 represents the pressure within the patient's breathing circuit, graph 624 represents the flow of gases within the patient's breathing circuit, graph 626 represents the volume of gases that has been introduced to the patient's breathing circuit through a ventilator, and graph 628 is an EIT plethysmograph of a dependent region of the patient's lungs. A reverse trigger (also referred to as “reverse triggering”; a type of asynchrony that occurs when a patient effort occurs after the initiation of a ventilator breath) occurs at a time indicated by line 621. Responsive to the reverse triggering, the impedance within the dependent region of the patient's lungs exhibits a positive deflection only during a second ventilation cycle, corresponding to line 621, and the volume graph 626 indicates breath stacking. The positive deflection of the impedance at time 623 illustrates a relationship between diaphragm contraction and the impedance within the dependent region. At time 625, the impedance within the dependent region decreases in response to an assisted cycle via the ventilator beginning at time 623, indicating that a reverse triggering has occurred and there is a patient-ventilator asynchrony. At time 625, the patient is not responding to ventilation provided by the mechanical ventilator.

Accordingly, a respiratory effort of a patient, a patient-ventilator asynchrony, or both may be detected (identified) by comparing impedance data from at least one dependent region of the lungs of a patient with one or more historical values of the impedance data of the at least one dependent region of the lungs, stored patterns of impedance data of the dependent region of the lungs, concurrent impedance data for a second region (at least a portion of which comprising a non-dependent region of the lungs) of the lungs of the patient, a concurrent flow of gases in the airway of the patient, and a concurrent pressure within the airway. Without being bound by any particular theory, it is believed that by analyzing the impedance of the dependent region of the lungs, the patient-ventilator asynchrony may be detected because the dependent region of the lungs is more responsive to the activity of the diaphragm than the non-dependent regions of the lungs. Accordingly, efforts to breathe by the patient (which being with the diaphragm) may be monitored via the impedance activity of the dependent region of the lungs.

In some embodiments, a relative increase in the impedance within the dependent region without one or more of a corresponding increase in the impedance within the second region, an increase in a flow of gases within the airway, or an increase in pressure within the airway may be an indication of patient-ventilator asynchrony, such as ineffective effort. Advantageously, the patient-ventilator asynchrony may be detected via non-invasive means, such as by the use of EIT through the application of voltages to a chest of the patient. In addition, the patient-ventilator asynchrony may be detected during use of a mechanical ventilator and an EIT system, and does not require additional equipment or invasive catheters. By way of contrast, conventional methods of determining patient-ventilator asynchrony include, for example, esophageal pressure tracing (which requires esophageal balloon) or neurally adjusted ventilator assist (NAVA), both of which require positioning of a catheter and are invasive techniques.

### Example

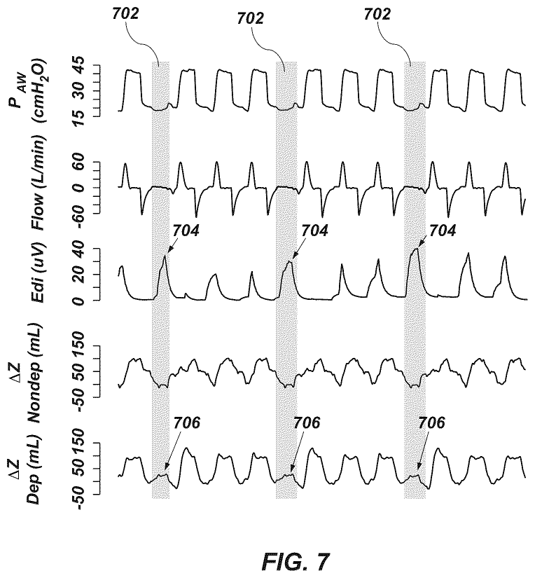

The detection of ineffective effort based on impedance data or image data obtained from the impedance data (EIT) for the dependent region of the lungs was verified using electrical activity of the diaphragm (Edi) monitoring in a piglet. FIG. 7 is a graph of flow and airway pressure measured at the airway of a piglet, and impendence data of the lungs, measured at the posterior and at the anterior regions, as well as graphs of the electrical activity of the diaphragm. Ineffective efforts are indicated in the shaded regions 702 of the graph.

The animal's respiratory effort can be observed by the increased electrical activity of the diaphragm (indicated at arrows 704) accompanied by a minor increase in the impedance of the dependent region of the lungs (indicated at arrows 706) at times corresponding to no increase in the airway pressure and no flow through the airway. Accordingly, the minor increase in the impedance of the dependent region at times during which there is no flow or no increase in flow or pressure in the airway may be indicative of an ineffective effort asynchrony.

Additional non-limiting example embodiments of the disclosure are set forth below.

Embodiment 1: A method of determining a patient's respiratory effort, the method comprising: acquiring first impedance data representative of a first region of lungs of a patient, the first region comprising at least a dependent region of the lungs; comparing the first impedance data with one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region; and based on the comparison, identifying the occurrence of patient's respiratory effort.

Embodiment 2: The method of Embodiment 1, wherein acquiring first impedance data representative of a first region of the lungs of the patient comprises acquiring the first impedance data of only a dependent region of the lungs.

Embodiment 3: The method of Embodiment 1 or Embodiment 2, wherein acquiring first impedance data representative of a first region of lungs of the patient comprises acquiring the first impedance data while the patient is in operable communication with a ventilator.

Embodiment 4: The method of any one of Embodiments 1 through 3, wherein comparing the first impedance data comprises comparing the first impedance data with the historical impedance data of the first region.

Embodiment 5: The method of any one of Embodiments 1 through 4, wherein comparing the first impedance data with one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises comparing the first impedance data with each of the second impedance data, the flow rate, and the pressure over the same time period.

Embodiment 6: The method of any one of Embodiments 1 through 5, wherein comparing the impedance data with one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises determining an ineffective effort of the patient.

Embodiment 7: The method of any one of Embodiments 1 through 6, wherein acquiring first impedance data representative of a first region of the lungs of the patient comprises acquiring the first impedance data representative of a dependent region and a non-dependent region of the lungs.

Embodiment 8: The method of any one of Embodiments 1 through 7, wherein acquiring first impedance data representative of a first region of the lungs of the patient comprises acquiring an EIT plethysmograph of the first region of the lungs.

Embodiment 9: The method of any one of Embodiments 1 through 8, further comprising determining an asynchrony between the patient and the ventilator responsive to measuring an increase in the first impedance without an increase in the pressure or the flow rate within the breathing circuit.

Embodiment 10: The method of any one of Embodiments 1 through 9, further comprising determining an asynchrony between the patient and the ventilator responsive to measuring an increase in the first impedance without a corresponding increase in the second impedance.

Embodiment 11: The method of any one of Embodiments 1 through 10, wherein comparing the first impedance data with one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises determining a reverse trigger event.

Embodiment 12: A system for determining a respiratory effort of a patient, the system comprising: an electrical impedance tomography system; at least one processor coupled to the electrical impedance tomography system; and at least one non-transitory computer readable storage medium storing instructions thereon that, when executed by the at least one processor, cause the at least one processor to: acquire, with the electrical impedance tomography system, first impedance data representative of at least one dependent region of lungs of a patient; and compare the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs to identify a respiratory effort of the patient based on the comparison.

Embodiment 13: The system of Embodiment 12, further comprising at least one gas monitoring sensor operably coupled to the system, the at least one gas monitoring sensor configured to measure one or both of the pressure and the flow rate within the breathing circuit.

Embodiment 14: The system of Embodiment 12 or Embodiment 13, wherein comparing the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs to identify a respiratory effort of the patient comprises determining an asynchrony between the patient and a ventilator responsive to measuring an increase in the first impedance without a corresponding increase in one or more of the second impedance, the flow rate, and the pressure within the breathing circuit.

Embodiment 15: The system of any one of Embodiments 12 through 14, wherein the instructions are further configured to cause the at least one processor to start a respiratory cycle responsive to identifying an asynchrony between the patient and a ventilator.

Embodiment 16: The system of any one of Embodiments 12 through 15, wherein comparing the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs comprises comparing the first impedance data with the second impedance data responsive to determining an increase in the first impedance without a corresponding increase in one or both of the pressure and the flow rate in the breathing circuit.

Embodiment 17: The system of any one of Embodiments 12 through 16, wherein acquiring, with the electrical impedance tomography system, first impedance data representative of at least one dependent region of lungs of a patient comprises acquiring the first impedance data of only a dependent region of the lungs.

Embodiment 18: The system of any one of Embodiments 12 through 17, wherein comparing the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs comprises comparing the first impedance data with each of the flow rate within the breathing circuit, the pressure within the breathing circuit, the second impedance data, and the historical impedance data of the first region.

Embodiment 19: A system for determining a respiratory effort of a patient, the system comprising: an electrical impedance tomography system; and a controller, wherein the electrical impedance tomography system is operably coupled to the controller, the controller comprising: at least one processor; and at least one non-transitory computer readable storage medium storing instructions thereon that, when executed by the at least one processor, cause the at least one processor to: cause the electrical impedance tomography system to acquire first impedance data of a first region of lungs of the patient, the first region comprising a dependent region of the lungs; compare the first impedance data with one or more of second impedance data representative of a non-dependent region of the lungs of the patient, a flow rate within a breathing circuit of the patient, a pressure within the breathing circuit, a historical impedance data of the first region, and stored patterns of impedance data of the first region; and identify a respiratory effort of the patient based on the comparison.

Embodiment 20: The system of Embodiment 19, further comprising a ventilator in communication with the patient and operably coupled to the controller, the ventilator configured to provide a respiratory cycle to the patient responsive to identifying the respiratory effort of the patient.

While embodiments of the disclosure may be susceptible to various modifications and alternative forms, specific embodiments have been shown by way of example in the drawings and have been described in detail herein. However, it should be understood that the disclosure is not limited to the particular forms disclosed. Rather, the disclosure encompasses all modifications, variations, combinations, and alternatives falling within the scope of the disclosure as defined by the following appended claims and their legal equivalents.

1. A method of determining a respiratory effort of a patient, the method comprising:
acquiring first impedance data representative of a first region of lungs of a patient, the first region comprising at least a dependent region of the lungs;
comparing an increase or decrease in the first impedance data with increases or decreases of one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region; and
based on the comparison, identifying an occurrence of a respiratory effort of the patient and determining if the respiratory effort is ineffective.

2. The method of claim 1, wherein acquiring first impedance data representative of a first region of the lungs of the patient comprises acquiring the first impedance data of only a dependent region of the lungs.

3. The method of claim 1, wherein acquiring first impedance data representative of a first region of lungs of the patient comprises acquiring the first impedance data while the patient is in operable communication with a ventilator.

4. The method of claim 1, wherein comparing the first impedance data comprises comparing the first impedance data with the historical impedance data of the first region.

5. The method of claim 1, wherein comparing the increase or decrease in the first impedance data with increases or decreases in one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises comparing the first impedance data with each of the second impedance data, the flow rate, and the pressure over the same time period.

6. The method of claim 1, wherein comparing the increase or decrease in the impedance data with increases or decreases in one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises determining an ineffective effort of the patient.

7. The method of claim 1, wherein acquiring first impedance data representative of a first region of the lungs of the patient comprises acquiring the first impedance data representative of a dependent region and a non-dependent region of the lungs.

8. The method of claim 1, wherein acquiring first impedance data representative of a first region of the lungs of the patient comprises acquiring an EIT plethysmograph of the first region of the lungs.

9. The method of claim 1, further comprising determining an asynchrony between the patient and a ventilator responsive to measuring an increase in the first impedance without an increase in the pressure or the flow rate within the breathing circuit.

10. The method of claim 1, further comprising determining an asynchrony between the patient and a ventilator responsive to measuring an increase in the first impedance without a corresponding increase in the second impedance.

11. The method of claim 1, wherein comparing the increase or decrease in the first impedance data with increases or decreases in one or more of a flow rate within a breathing circuit of the patient, a pressure within the breathing circuit, a second impedance data representative of a second region of the lungs of the patient comprising at least a non-dependent region of the lungs of the patient, historical impedance data of the first region, and stored patterns of impedance data of the first region comprises determining a reverse trigger event.

12. A system for determining a respiratory effort of a patient, the system comprising:
an electrical impedance tomography system;
at least one processor coupled to the electrical impedance tomography system; and
at least one non-transitory computer readable storage medium storing instructions thereon that, when executed by the at least one processor, cause the at least one processor to:
acquire, with the electrical impedance tomography system, first impedance data representative of at least one dependent region of lungs of a patient;
receive a flow rate from within a breathing circuit of the patient and a pressure from within the breathing circuit; and
compare an increase or decrease in the first impedance data with an increase or decrease in one or more of the flow rate within the breathing circuit of the patient, the pressure within the breathing circuit, a second impedance data representative of a second region of the lungs of the patient comprising at least a non-dependent region, historical impedance data of the first region, and stored patterns of impedance data representative of the first region of the lungs to identify a respiratory effort of the patient based on the comparison and determine if the respiratory effort is ineffective.

13. The system of claim 12, further comprising at least one gas monitoring sensor operably coupled to the system, the at least one gas monitoring sensor configured to measure one or both of the pressure and the flow rate within the breathing circuit.

14. The system of claim 12, wherein comparing the increase or decrease in the first impedance data with the increase or decrease of one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs to identify a respiratory effort of the patient comprises determining an asynchrony between the patient and a ventilator responsive to measuring an increase in the first impedance without a corresponding increase in one or more of the second impedance, the flow rate, and the pressure within the breathing circuit.

15. The system of claim 12, wherein the instructions are further configured to cause the at least one processor to send a command to start a respiratory cycle responsive to identifying an asynchrony between the patient and a ventilator.

16. The system of claim 12, wherein comparing the first impedance data with one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs comprises comparing the first impedance data with the second impedance data responsive to determining an increase in the first impedance without a corresponding increase in one or both of the pressure and the flow rate in the breathing circuit.

17. The system of claim 12, wherein acquiring, with the electrical impedance tomography system, first impedance data representative of at least one dependent region of lungs of a patient comprises acquiring the first impedance data of only a dependent region of the lungs.

18. The system of claim 12, wherein comparing the increase or decrease in the first impedance data with the increase or decrease in the one or more of a flow rate within a breathing circuit of the patient, a pressure within the breathing circuit, a second impedance data representative of a second region of the lungs of the patient comprising at least a non-dependent region, historical impedance data of the first region, and stored patterns of impedance data representative of the first region of the lungs comprises comparing the first impedance data with each of the flow rate within the breathing circuit, the pressure within the breathing circuit, the second impedance data, and the historical impedance data of the first region.

19. A system for determining a respiratory effort of a patient, the system comprising:
an electrical impedance tomography system; and
a controller, wherein the electrical impedance tomography system is operably coupled to the controller, the controller comprising:
at least one processor; and
at least one non-transitory computer readable storage medium storing instructions thereon that, when executed by the at least one processor, cause the at least one processor to:
cause the electrical impedance tomography system to acquire first impedance data of a first region of lungs of the patient, the first region comprising a dependent region of the lungs;
receive a flow rate from within a breathing circuit of the patient and a pressure from within the breathing circuit;
compare an increase or decrease in the first impedance data with an increase or decrease in one or more of second impedance data representative of a non-dependent region of the lungs of the patient, the flow rate within the breathing circuit of the patient, the pressure within the breathing circuit, a historical impedance data of the first region, and stored patterns of impedance data of the first region; and
identify a respiratory effort of the patient and determine if the respiratory effort is ineffective based on the comparison.

20. The system of claim 19, further comprising a ventilator in communication with the patient and operably coupled to the controller, the ventilator configured to provide a respiratory cycle to the patient responsive to identifying the respiratory effort of the patient.
